(19) United States
(12) Patent Application Publication
(10) Pub. No.: US 2020/0345991 A1
(43) Pub. Date: Nov. 5, 2020

(54) JACKETED CATHETER PROBES AND METHODS OF USE FOR INFUSION

(71) Applicant: The USA, as represented by the Secretary, Department of Health and Human Services, Bethesda, MD (US)

(72) Inventors: John A. Chiorini, Bethesda, MD (US); Blake M. Warner, Bethesda, MD (US); Ilias Alevizos, Bethesda, MD (US)

(21) Appl. No.: 16/758,636

(22) PCT Filed: Oct. 26, 2018
(86) PCT No.: PCT/US2018/057744

§ 371 Date: Apr. 23, 2020

Related U.S. Application Data
(60) Provisional application No. 62/577,389, filed on Oct. 26, 2017.

(51) Int. Cl. A61M 29/00; A61M 25/01

(52) U.S. Cl. CPC A61M 29/00; A61M 2025/0687; A61M 25/0136

(57) ABSTRACT

This disclosure generally relates to devices, systems, or kits of color-coded dilation probes and corresponding sheath or jacket catheters that dilate tissue in an atraumatic manner to a desired depth and diameter for the insertion of a cannula. A jacketed catheter may be engaged to the probes and inserted into the dilated tissue to form a fluid-tight seal. An infusate is then introduced into the tissue through the catheter and held in the tissue for a desired period of time.

### CROSS-REFERENCE TO RELATED APPLICATIONS

The present disclosure claims priority to U.S. Provisional Application No. 62/577,389, entitled “Jacketed Catheter Probes and Methods of Use for Infusion,” filed on Oct. 26, 2017; the entire contents of which are incorporated by reference it its entirety.

### GOVERNMENT INTEREST STATEMENT

The present subject matter was made with U.S. government support. The U.S. government has certain rights in this subject matter.

### FIELD

The present disclosure relates to systems, methods, and devices for accessing and dilating soft tissue, such as a gland, using a jacketed probe. Further, the present disclosure relates to methods of using the jacket probe to infuse a solution into the soft tissue.

### BACKGROUND

Salivary gland duct cannulation and retrograde infusion are clinical practices sometimes used in medical imaging. Cannulation requires expert training and long appointment times to ensure clinical success. The difficulty of carrying out these methods has been a barrier to widespread clinical acceptance. Furthermore, most catheters are clear or translucent with few markings so it is only possible to estimate how deep into the duct a catheter is placed. This uncertainty also lends itself to unsecured catheter placements that may result in an infusate leaking from the catheter or duct.

While improvements in saliva duct endoscopy for the treatment of salivary stones have been made, these procedures are often complicated, invasive and require extensive ductal dilation with specialized tools that require local or general anesthesia. These conditions greatly increase the procedural time, risk of duct injury, risk of complications, along with requiring expensive equipment and extensive training. As such, a need exists for a straightforward, quick, atraumatic system and method to dilate and infuse soft tissue without anesthesia that takes advantage of the existing soft tissue anatomy to prevent infusate leakage.

### SUMMARY

The present disclosure generally relates to a jacketed probe system, kit, and methods for dilating and infusing a fluid into soft tissue. In one embodiment, a jacketed probe kit for atraumatic gland infusion includes at least one solid double-ended dilator probe, further including a tapered distal end and a tapered proximal end engaged to a central portion. The central portion of the solid double-ended dilator probes has a unique outer diameter in a range between about 0.75 mm and about 1.25 mm. The tapered distal end has a length in a range between about 40 mm and about 80 mm and the tapered proximal end has a length in a range between about 20 mm and about 40 mm. The tapered distal end and the tapered proximal ends are configured for atraumatic dilation of tissue. The kit also includes at least one catheter that defines a cannula for tissue infusion. The catheter has a proximal catheter end and a distal catheter end. The catheter corresponds to the at least one dilator probe and has an inner diameter equal to the unique outer diameter of the central portion of the dilator probe. The tapered distal end of the dilator probe is received in the catheter to dilate a gland to a desired depth and to aid insertion of the cannula into the gland. The tapered proximal end of the dilator probe is received in the gland to dilate the gland to a desired width.

According to one embodiment, a method for gland infusion access using a jacketed probe kit having a plurality of dilation probes includes determining the desired depth and the desired width for the gland infusion access site. Next, a depth dilation probe having at least one end equal to the desired depth is selected. A first end of the depth dilation probe is inserted into an elongated catheter, wherein the first end of the depth dilation probe extends distally beyond a distal end of the elongated catheter. The first end of the depth dilation probe is rotated and advanced to dilate a gland to a desired depth. The first end of the depth dilation probe is removed from the elongated catheter and a width dilation probe having at least one end equal to the desired width is selected. The method further includes inserting a second end of the width dilation probe into the elongated catheter, wherein the second end of the width dilation probe has an outer diameter greater than that of the first end of the depth dilator probe. The second end of the width dilation probe extends distally beyond the distal end of the elongated catheter. The second end of the width dilation probe is rotated and advanced to dilate the gland to a desired width and defining a dilated orifice. The second end of the width dilation probe from the elongated catheter is removed and the first end of the depth dilation probe is reinserted into the elongated catheter. The first end of the depth dilation probe and the elongated catheter is advanced into the dilated orifice, and then the first end of the depth dilation probe is removed from the catheter and orifice. The elongated catheter is secured in the dilated orifice a fluid is infused into the gland.

In yet another embodiment, jacketed probe set may include a plurality of solid double-ended dilator probes, each further including a tapered distal end and a tapered proximal end engaged to a central portion. The central portion of each of the plurality of solid double-ended dilator probes has a unique outer diameter in a range between about 0.75 mm and about 1.25 mm. The tapered distal end has a length in a range between about 40 mm and 80 mm and the tapered proximal end has a length in a range between about 20 mm and about 40 mm. The tapered distal end and the tapered proximal ends are configured for atraumatic dilation of tissue. The kit also includes a plurality of catheters each defining a cannula for tissue infusion and each having a proximal catheter end and a distal catheter end. Each of the plurality of catheters corresponds to a particular dilator probe and has an inner diameter equal to the unique outer diameter of the central portion of the corresponding dilator probes. The tapered distal end of a first dilator probe is received in a first corresponding catheter to dilate a gland to a desired depth and to insert the cannula into the gland. The tapered proximal end of the first dilator probe is received in the first corresponding catheter to dilate the gland to a desired width.

Additional objectives, advantages, and novel features will be set forth in the description that follows or will become apparent to those skilled in the art upon examination of the drawings and detailed description that follows.

### BRIEF DESCRIPTION OF THE DRAWINGS

FIG. 1 is an illustration of a small probe of the jacketed probe kit, according to one embodiment. The small probe is shown with and without the jacketed catheter.

FIG. 2 is an illustration of a medium probe of the jacketed probe kit, according to one embodiment. The medium probe is shown with and without the jacketed catheter.

FIG. 3 is an illustration of a large probe of the jacketed probe kit, according to one embodiment. The large probe is shown with and without the jacketed catheter.

FIG. 4 is an illustration of a jacketed probe kit according to one embodiment.

FIG. 5 is a table identifying dimensions for the probes and jacketing catheters according to one embodiment.

FIG. 6 is a flowchart of a method for using a jacketed probe kit to access and infuse soft tissue according to one embodiment.

Reference characters indicate corresponding elements among the views of the drawings. The headings used in the figures do not limit the scope of the claims.

Any dimensions presented in the figures are merely for example and do not necessarily limit the size of the devices depicted.

### DETAILED DESCRIPTION

The present disclosure generally relates to a device, system, or kit of color-coded dilation probes and corresponding sheath or jacket catheters that may dilate tissue in an atraumatic manner to a desired depth and dilation for the insertion of a cannula. In various aspects, the sheath or jacket catheter may also function as the cannula, once the dilation probe is withdrawn. After the cannula is secured, a fluid may be infused into the tissue. A desired advantage of the present system is that the orifice is dilated to precise dimensions to for a fluid tight-seal around the inserted cannula. As such, the infused solution may be easily retained within the tissue for a desired period of time without leakage, thus ensuring that the desired dosage is received within the tissue.

In one aspect, the devices, systems, and methods disclosed herein may be used for localized salivary gland therapies such as gene therapy vector delivery to the gland or the addition of inhibitors to the salivary gland to protect them from radiation damage during the treatment for head and neck cancers. In another aspect, the devices, systems, and kits disclosed herein may be used to deliver a solution with a gene therapy drug for radiation induced xerostomia or other drugs that could be infused that you would also want to remain in the gland for a period of time. The infusate may include radioprotectants to prevent damage to the gland during radiation therapy for cancer, an anti-inflammatory agent of the treatment of Sjogren's or acute sialadenitis, or stem cells or cell suspension for regenerating a salivary gland. As salivary glands are natural secretory organs that may further function as a bioreactor for the infused gene therapy vectors and produce proteins that enter the bloodstream for treating hormone deficiencies or systemic diseases. Alternatively, the therapies could increase saliva production to aid in digestion, enhance anticaries agents, combat periodontal disease, upper gastrointestinal problems, or infection. The disclosed devices are useful for these applications and others, where a solution is delivered locally to a gland (e.g., parotid, submandibular, and/or sublingual) and held in the gland for a desired period of time.

The system includes a plurality of catheters and a corresponding plurality of tapered dilation probes. The catheters may be provided as a set of commonly used sizes (e.g. 20-24 gauge). Similarly, the dilation probes are provided in a corresponding set, where the dilation probes vary in diameter and dilation depth and protrude from a distal end of the catheter. The probes and catheters are presented in a color-coded arrangement, so a user may easily select the appropriate probe and catheter combination for the desired dilation. Typically, the appropriate catheter size is selected, and then one or more suitable dilation probes that correspond to the selected catheter are selected. The color-coded markings permit a user to observe the depth of dilation and, in various aspects, the markings function as radiopaque markers to visualize the catheter and/or probes during radiographic examination. Additionally, the color-coded system permits easy visualization during infusion to limit or prohibit anterior infusate loss, displacement of the cannula, or loss of seal at the duct orifice. These visual aids further make the probe system suitable for use in non-dental setting or lower light settings where sufficient overhead lighting is limited.

When in use the catheter/probe combinations are used in various sequences to dilate glands, such as the submaxillary or parotid glands, to a specific depth and diameter. The diameter of dilation is selected to match the desired diameter of the catheter or another cannula that will be positioned within the gland closely to provide a fluid seal for the infusion. Similarly, the depth of dilation is precisely measured to correspond to the parameters of the infusion protocol. It may be necessary to use many probes to achieve the desired depth and diameter.

An added benefit of this system will minimize intra-procedural loss of infusate. The internal diameter of the duct, through the dilation process, is size-matched to the external diameter of the cannula. This relationship ensures an adequate seal is created between the duct and cannula interface to prevent forward loss of infusate. Moreover, intravenous glycopyrolate shuts down basal saliva flow and has the added benefit of slight narrowing of the excretory duct (which promotes a better seal on the cannula, potentially). A method of using the system to infuse a gland may include dilating the gland to a desired depth using a first probe inserted into the catheter. The dilation may be performed by rotating the probe about its longitudinal axis while advancing the probe into the gland. After dilation to an optimal depth, the first probe is removed from the catheter and a second probe is inserted into the initially dilated portion gland to further dilate the duct. One or more secondary probes may be used sequentially to obtain the desired orifice diameter. Similarly, the probe is advanced into the gland, while rotating the second probe about its longitudinal axis. The second probe is removed from the catheter and the first probe is then reinserted into the catheter and inserted back into the fully dilated gland orifice. The distal end of the catheter is advanced into the probed orifice to the desired depth. The first probe is removed, leaving the catheter in the gland. The catheter is secured into the gland and a fluid source is engaged to the proximal end. The infusion protocol is then performed.

While the systems and methods disclosed herein are described in relation to the dilation and probing of glands, such as but not limited to Stenson's gland (parotid gland) or Wharton's ducts (submandibular glands), the probes and methods may be used to dilate other soft tissue structures.

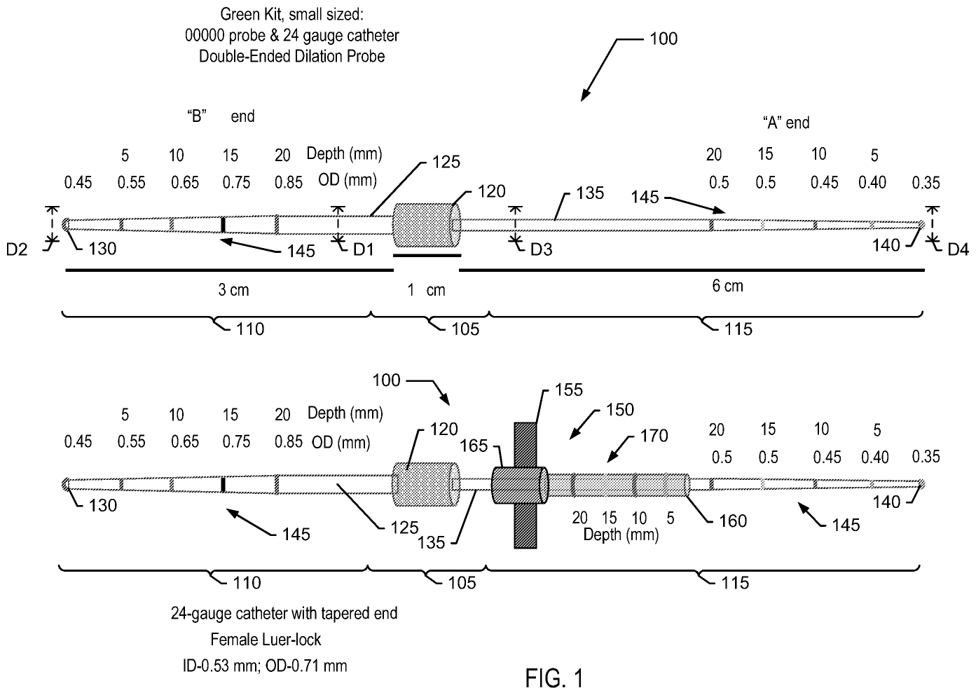

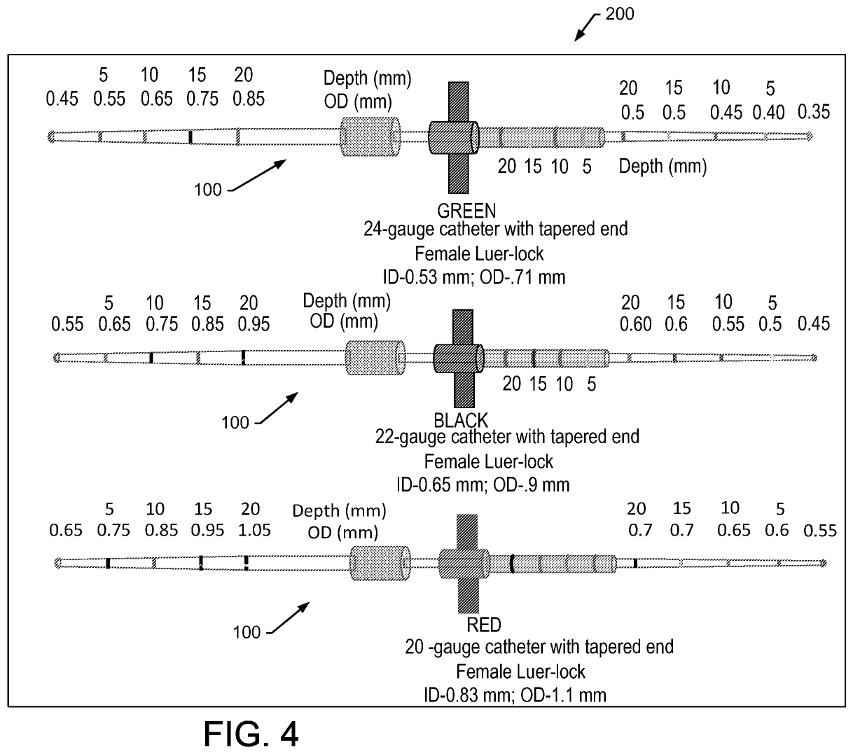

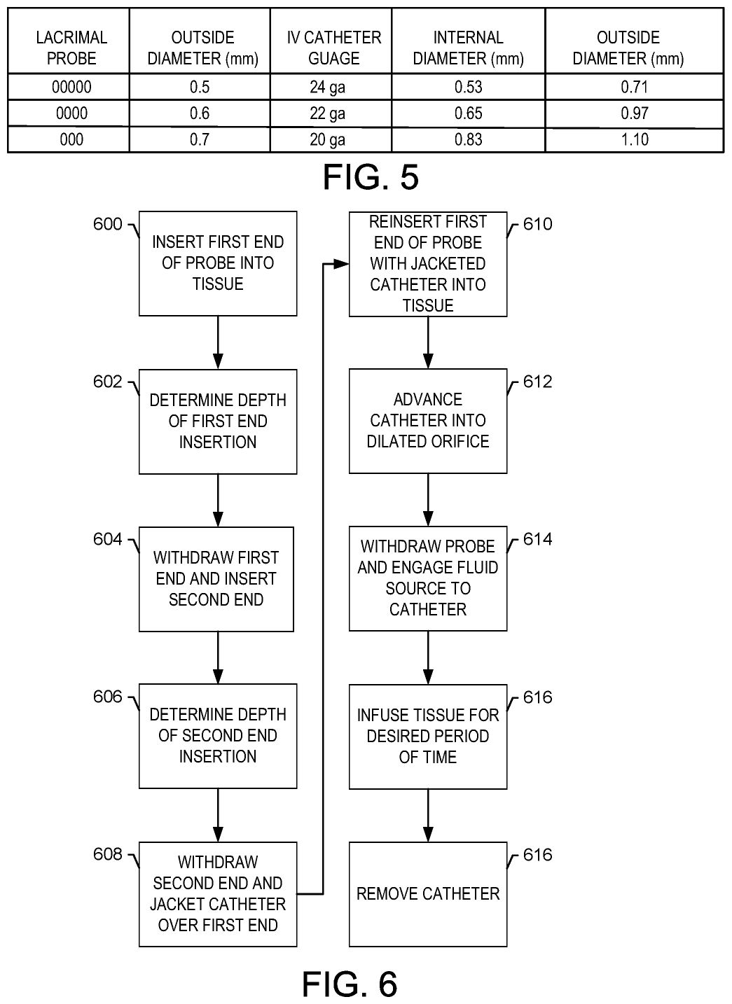

FIGS. 1-3 show embodiments of the dilating probe 100 alone and in a single probe and catheter kit 200. A multiple probe kit 300, as shown in FIG. 4, includes at least one probe 100 and a corresponding catheter 150. In one embodiment, the multiple probe kit 300 includes at least one small, medium, and large probe 100 and corresponding catheters 150.

The probe 100 is a dual-ended probe having a central region 105 engaged to a tapered proximal end 110 and a tapered distal end 115. According to various embodiments, the central region 105 may include a handle 120. The handle 120 may be knurled, textured, or otherwise configured to provide an easily graspable surface. According to one aspect, each probe 100 may be identified as small, medium, or large and may be identified by a lacrimal size of 00000, 0000, and 000, respectively.

The probe 100 may be composed of any suitable material. In one embodiment, the probe 100 is composed of a low-friction resin or polymer and provided as a sterile single-use disposable probe. In another embodiment, the probe 100 may be a reusable and autoclavable probe composed of a metal or metal alloy. Regardless of composition, it is preferable that the probe 100 is semi-flexible such that the probe has sufficient compressive strength to dilate the tissue, yet flexible enough to navigate tortuous paths and cavities. In one embodiment, the flexibility of the probe 100 may vary along the length of the probe with the proximal and distal tips 130 and 140, respectively, having greater flexibility. Portions of the probes may be made of flexible or semi-flexible resin or polymers (e.g., nylon, polyethylene, copolymer, polypropylene, PVC, Teflon, polyamide, or any other suitable plastic material) to achieve specified performance characteristics. In various embodiments, portions of the probe may be composed of commercially available medical grade resin polymers that are selected for desired performance characteristics, such as but not limited to strength, flexibility, autoclavability, low friction, or combinations thereof.

As used herein, tapered refers to a metered incremental increase or decrease in the diameter of the proximal end 110 and the distal end 115. In one embodiment, the proximal end 110, the distal end 115, or both have a continuous taper. In another embodiment, the proximal end 110, the distal end 115, or both have a stepped taper.

In one embodiment, the probe 100 may have an overall length of 10 cm, however in other embodiments; the probe may have an overall length of 6 cm to 20 cm. In one embodiment, the proximal end 110 may have a length of approximately 3 cm, while the central region 105 may have a length of approximately 1 cm, while the distal end 115 has a length of approximately 6 cm.

The tapered proximal end 110 has a maximum diameter D1 at a proximal catheter portion 125 adjacent to the central region 105 and a minimum diameter D2 at a proximal terminal tip 130. Similarly, the tapered distal has a maximum diameter D3 at a distal catheter portion 135 adjacent to the central region 105 and a minimum diameter D4 at a distal terminal tip 140. As shown, the maximum diameter D1 of the proximal end 110 is greater than the maximum diameter D3 of the distal end 115. In various embodiments, the maximum diameter D3 of the distal end 115 is greater than the minimum diameter D2 of the proximal end 110.

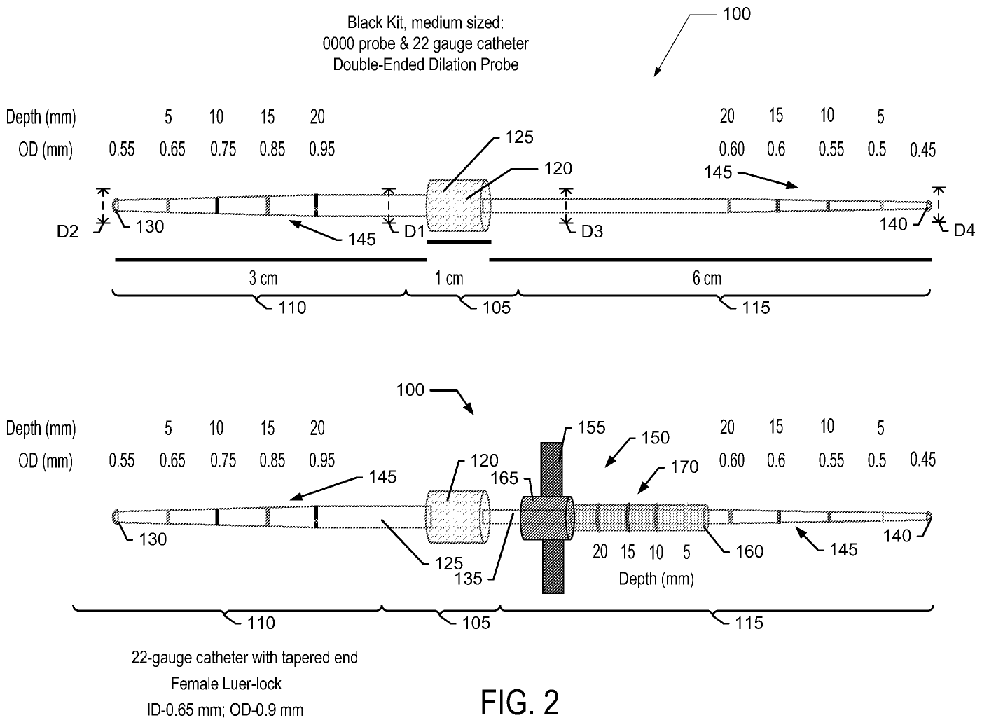

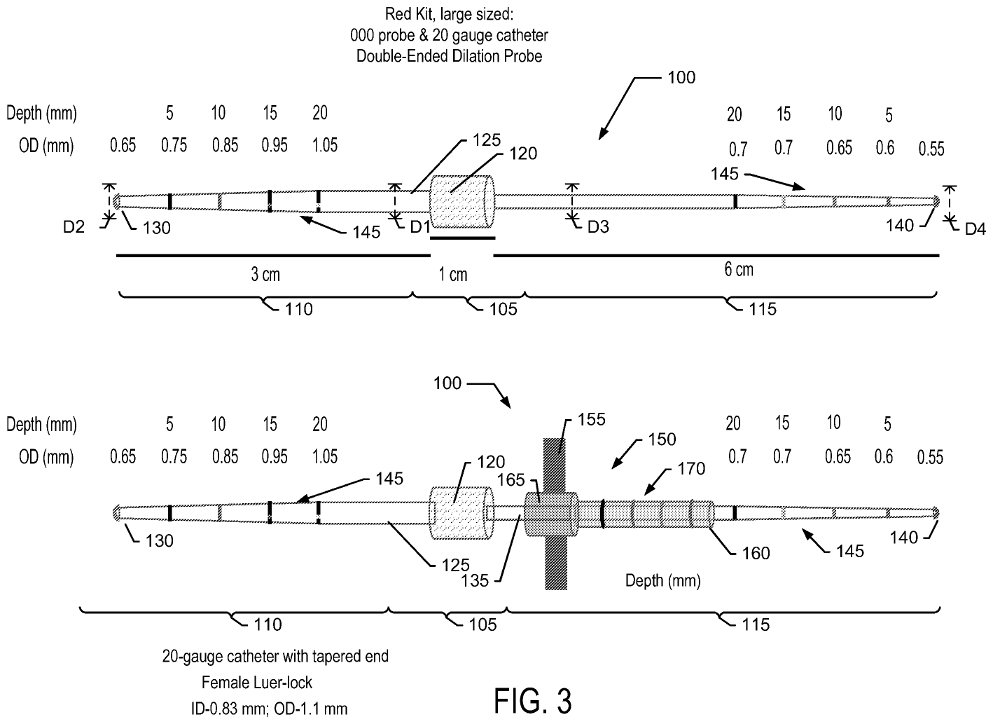

As shown in FIG. 1, the tapered proximal end 110 for a small probe, having a lacrimal size of 00000, has an outer diameter in a range from 0.45 mm to 0.85 mm, while the tapered distal end 115 has an outer diameter in a range from 0.35 mm to 0.5 mm. Similarly, the tapered proximal end 110 for a medium probe, having a lacrimal size of 0000 and shown in FIG. 2, has an outer diameter in a range from 0.55 mm to 0.95 mm, while the tapered distal end 115 has an outer diameter in a range from 0.45 mm to 0.6 mm. The tapered proximal end 110 for a large probe, having a lacrimal size of 000 and shown in FIG. 3, has an outer diameter in a range from 0.65 mm to 1.05 mm, while the tapered distal end 115 has an outer diameter in a range from 0.55 mm to 0.7 mm. As such, the multiple probe kit of FIG. 4, provides the ability to dilate soft tissue or a gland to an orifice diameter between 0.35 mm and 1.05 mm at a depth between 5 mm and 20 mm

As noted in FIGS. 1-4, the tapered distal end 115 may be referred to as the “A” end, while the tapered proximal end 110 may be referred to as the “B” end. The “A” end of the probe has the smallest minimum diameter D4 and are preferably used initially to probe the desired tissue and advanced to reach a desired depth. Conversely, the “B” end has a greater maximum diameter D1 and is preferably used dilate the tissue to a desired orifice diameter after the initial probing with the “A” end. The orifice diameter is approximately equal to the outer diameter of the selected jacketing catheter 150.

As shown, the tapered proximal end 110 and a tapered distal end 115 also include a series of markings 145 that correspond to a linear length of the probe ends. The markings 145 provide an easily discernable indication of the depth of placement when the probe end is inserted in to the soft tissue. In one aspect, the markings 145 are color-coded, while in another aspect the markings may include other indicia, including but not limited to hatch marks, tick marks, or alphanumeric symbols, to distinguish the sections of the tapered ends. For purposes of illustration only, an example color coding scheme may be:
0.45 mm—purple
0.50 mm—yellow
0.55 mm—red
0.60 mm—blue
0.65 mm—green
0.70 mm—gray
0.75 mm—black
0.80 mm—pink
0.85 mm—purple
0.90 mm—orange
0.95 mm—red/black
1.05 mm—yellow/black

In another embodiment, the paint used on the catheter could be differentially radiopaque. During sialography, users can know exactly where in the duct the catheter is. If there are strictures in the duct, you would know exactly how deeply into the duct the stricture lies and if, in fact, it can be dilated (a routine procedure for ductal strictures).

The proximal catheter portion 125 and the distal catheter portion 135 are elongated non-tapered portions of the proximal end 110 and distal end 115, respectively. When assembled with a jacketing catheter 150, the proximal catheter portion 125 and the distal catheter portion 135 serve as staging areas for the catheter. As described more fully below, the catheter 150 is held on the proximal catheter portion 125 or the distal catheter portion 135, while the desired tissue is dilated.

In various embodiments, the jacketing catheter 150 is a sterile, flexible, disposable catheter that is sized to match the dilation probe 100. By way of example and not limitation, the catheter 150 may be an intravenous (“IV”) 20 gauge, 22 gauge, or 24 gauge catheter that corresponds to the size of the dilation probes 100, shown in FIGS. 1-3. However, consistent with the spirit of the present disclosure, the probe and catheter sizes may be larger or smaller and configured for use on anatomically larger or smaller ducts in humans and other larger animal models. As such, on other embodiments, the catheter may be in a range between and including 16 gauge IV catheters up to 26 gauge IV catheters. Similarly, larger, smaller, longer, and shorter probes 100 may be used in conjunction with the catheters to accommodate anatomic variation in human and animal patients.

In one aspect, the catheter 150 may be between 10 to 50 mm in length. The catheter 150 may include a handle or flange portion 155 that may be engaged with forceps or hemostats to maneuver and retain the catheter in a desired position. In various embodiments, the flange portion 155 may include one or more holes for grasping, adhering, and/or suturing the catheter in place to facilitate longer-term stable placement in the duct.

The catheter may also include a tapered leading edge 160 and a luer-lock or luer-slip fitting 165 to engage a fluid source to the catheter after placement within dilated tissue. Similar to the probe 100, the catheter 150 may also include a set of color-coded or other identifiable markings 170 to provide easily discernable indications of the length of the catheter. The markings 170 may also be used to determine a depth of placement within dilated tissue.

### EXAMPLE METHODS OF PROBING, DILATING, AND INFUSING SOFT TISSUE

A method of probing, dilating, and infusing soft tissue is shown in the flowchart of FIG. 6. At 600, a first end of a sterile probe is inserted into the tissue to the desired depth. The probe may be rotated in and alternating clockwise and counter-clockwise manner as it is advanced into the tissue. At 602, the probe is observed to determine if the desired depth of dilation has been reached. This may be determined by visual observation of markings on the first end of the probe. The probe is withdrawn and the second end of the probe is inserted into the tissue at 604. The second end of the probe is used to dilate the tissue to a desired orifice diameter. At 606, the probe is observed to determine if the desired orifice diameter has been achieved. This may be determined by visual observation of markings on the first end of the probe. At 608, the second end of the probe is withdrawn and an infusion catheter is jacketed over the first end. The first end of the probe is reinserted into the tissue at 610 and the catheter is advanced forward into the dilated orifice at 612, forming fluid-tight seal. The catheter may be held in place while the probe is removed and a fluid source is engaged to the catheter at 614. The desired infusate is provided through the catheter to the tissue and 616 for a desired period of time. Lastly, the catheter is removed from the tissue at 618. In various embodiments, the first end or the second end of the probe may be used to insert the catheter 614. As such, the catheter 614 may be positioned on the “A” end or the “B” end of the probe.

In another embodiment, a method of infusion includes providing an antiseptic rinse, such as but not limited to 0.12% chlorhexidine solution prior to cannulation. The rinse may be applied for 15 seconds to 45 seconds. Next, on a sterile drape, a selected probe, such as a medium probe kits, an embodiment of which is shown in FIG. 2, may be opened. After donning sterile gloves, the probe is removed from the packaging and the jacketed catheter may be removed from the “A” end of the probe, and placed back on the sterile drape. An orifice of the duct to be dilated is identified and a small puncta is visualized on the top/side of the duct, such as the parotid papilla. The “A” end of the probe is used to gently explore and probe the duct orifice using a gentle back and forth rotating motion. Attention is paid to how deeply the probe is advanced into the duct, by using the visual indicators, or color bands on the probe. For example, if the probe is advanced into the duct to, or slightly past, the PURPLE band. The probe is withdrawn and rotated so that the “B” end of the probe may be used to further explore and dilate the duct orifice to the BLACK or PURPLE band using the same gentle rotating motion. Once the desired depth is reached, the “B” end is withdrawn and the sterile catheter is jacketed back on the “A” end of the probe. The “A” end of the probe is advanced back into the duct to the PURPLE band. Then the catheter is advanced into the duct manually or with a tool such as hemostats or forceps. The catheter is advanced until the desired colored marker band is at the surface of the orifice, for example, BLACK, PURPLE, or RED/BLACK which may correspond to a depth of 1.0, 1.5, or 2.0 cm, respectively. At the desired depth, the probe is removed, while the catheter is held in place. The fluid source is attached to the catheter and the desired solution is infused into the tissue.

In yet another experimental method, a probe of 0000 lacrimal diameter was placed in the sheath of a 22 gauge radiopaque catheter. The tip of the probe was extended approximately 1 mm beyond the end of the sheath to create a 0000 size jacketed catheter probe. The 0000 jacketed catheter probe was then gently advanced into the duct orifice approximately 1-1.5 cm posteriorly. With the catheter jacket held in place by forceps the probe was removed from the jacket and a luer-lock syringe containing the vector was attached and held in place. With the catheter held in place, approximately 2 μg/kg of glycopyrrolate was administered intravenously to arrest existing salivary flow and prevent interference with vector transduction. After approximately 10 minutes, the oral cavity was inspected to determine the arrest of salivary flow. Suspended in a fluid vehicle, the specified dose of AAV2-AQP1 was then infused into the targeted parotid gland in a retrograde direction using the predetermined gland infusate volume. The cannula was then held in place for 10 minutes to ensure reasonable contact time between the vector and gland epithelia, and to prevent anterior loss of the infusate. Following removal of the cannula, the draining AAV2-AQP1 suspension was suctioned along the buccal mucosa, vestibule, and floor of mouth for five minutes.

It should be understood from the aforementioned descriptions that while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. An atraumatic tissue infusion access kit comprising:
at least one solid double-ended dilator probe, further comprising:
a tapered distal end and a tapered proximal end engaged to a central portion;
wherein the central portion of the solid double-ended dilator probe has a unique outer diameter in a range between 0.35 mm and 1.25 mm,
wherein the tapered distal end has a length in a range between 40 mm and 80 mm and the tapered proximal end has a length in a range between 20 mm and 40 mm; and
wherein the tapered distal end and the tapered proximal ends are configured for atraumatic dilation of tissue;
at least one catheter, defining a cannula for tissue infusion and having a proximal catheter end and a distal catheter end; wherein the catheter corresponds to the at least one dilator probe and has an inner diameter equal to the unique outer diameter of the central portion of the dilator probe;
wherein the tapered distal end of the dilator probe is received in the catheter to dilate the tissue to a desired depth and to insert the cannula into the tissue; and
wherein the tapered proximal end of the dilator probe is received in the catheter to dilate the tissue to a desired width.

2. The tissue infusion access kit of claim 1 wherein the dilator probe extends distally beyond the elongated catheter by approximately 1 cm to 1.5 cm.

3-5. (canceled)

6. The tissue infusion access kit of claim 1 wherein the tapered proximal end has an outer diameter greater than that of the tapered distal end.

7. The tissue infusion access kit of claim 1 wherein the elongated catheter is identified by a plurality of color marker bands.

8. The tissue infusion access kit of claim 1 wherein the elongated dilator probe include a plurality of color marker bands.

9. The tissue infusion access kit of claim 8 wherein at least one of the plurality of color marker bands is radio opaque.

10. The tissue infusion access kit of claim 1, wherein at least one of the tapered distal end and the tapered proximal ends further comprises a semi-flexible material.

11. The tissue infusion access kit of claim 1, wherein the at least one catheter is in a range between a 16-gauge to 24-gauge catheter and wherein the distal catheter end has an outer diameter in a range between 0.5 and 1.8 mm.

12. A method for tissue infusion access using a tissue infusion access kit having a plurality of dilation probes; the method comprising:
determining the desired depth and the desired width for the tissue infusion access site;
selecting a depth dilation probe having at least one end equal to the desired depth;
inserting a first end of the depth dilation probe into an elongated catheter, wherein the first end of the depth dilation probe extends distally beyond a distal end of the elongated catheter;
rotating and advancing the first end of the depth dilation probe to dilate a tissue to a desired depth;
removing the first end of the depth dilation probe from the elongated catheter;
selecting a width dilation probe having at least one end equal to the desired width;
inserting a second end of the width dilation probe into the elongated catheter, wherein the second end of the width dilation probe has an outer diameter greater than that of the first end of the depth dilator probe, and wherein the second end of the width dilation probe extends distally beyond the distal end of the elongated catheter;
rotating and advancing the second end of the width dilation probe to dilate the tissue to a desired width and defining a dilated orifice;
removing the second end of the width dilation probe from the elongated catheter;
reinserting the first end of the depth dilation probe into the elongated catheter;
advancing the first end of the depth dilation probe and the elongated catheter into the dilated orifice;
removing the first end of the depth dilation probe;
securing the elongated catheter in the dilated orifice; and
infusing a fluid into the tissue.

13. The method of claim 12, wherein the depth dilator probe and the width dilator probe are configured as opposite ends of a dual-ended probe, wherein the depth dilator probe and width dilator probe are engaged to a central portion of the probe.

14. The method of claim 13, wherein the central portion comprises a handle.

15. The method of claim 12, wherein the infused fluid is retained in the tissue for a desired period of time.

16. The method of claim 15, wherein the desired period of time is greater than one hour.

17. An atraumatic tissue infusion access kit comprising:
a plurality of solid double-ended dilator probes, each further comprising:
a tapered distal end and a tapered proximal end engaged to a central portion;
wherein the central portion of each of the plurality of solid double-ended dilator probes has a unique outer diameter in a range between 0.75 mm and 1.25 mm,
wherein the tapered distal end has a length in a range between 40 mm and 80 mm and the tapered proximal end has a length in a range between 20 mm and 40 mm; and
wherein the tapered distal end and the tapered proximal ends are configured for atraumatic dilation of tissue;
a plurality of catheters, defining a cannula for tissue infusion and each having a proximal catheter end and a distal catheter end; wherein each of the plurality of catheters corresponds to a particular dilator probe and has an inner diameter equal to the unique outer diameter of the central portion of the corresponding dilator probes;
wherein the tapered distal end of a first dilator probe is received in a first corresponding catheter to dilate the tissue to a desired depth and to insert the cannula into the tissue; and
wherein the tapered proximal end of the first dilator probe is received in the first corresponding catheter to dilate the tissue to a desired width.

18. The tissue infusion access kit of claim 17 wherein the first dilator probe extends distally beyond the first elongated catheter by approximately 1 cm to 1.5 cm.

19-21. (canceled)

22. The tissue infusion access kit of claim 17 wherein the second dilator probe has an outer diameter greater than that of the first dilator probe.

23. The tissue infusion access kit of claim 17 wherein each of the plurality of elongated catheters is identified by a color marker band.

24. The tissue infusion access kit of claim 17 wherein each of the plurality of elongated dilator probes is identified by a color marker band.

25. The tissue infusion access kit of claim 24 wherein the color marker band is radio opaque.

26. The tissue infusion access kit of claim 17, wherein the central portion of each of the plurality of solid double-ended dilator probes further comprises a handle.
